(19) United States
(12) Patent Application Publication
(10) Pub. No.: US 2026/0183358 A1
(43) Pub. Date: Jul. 2, 2026

(54) COMPOSITION FOR HANGOVER RELIEF AND LIVER FUNCTION IMPROVEMENT, COMPRISING RAW POWDER MATERIAL, AND PREPARATION METHOD THEREFOR

(71) Applicant: Sang Ouk HA, Seoul (KR)

(72) Inventor: Sang Ouk HA, Seoul (KR)

(21) Appl. No.: 19/130,149

(22) PCT Filed: Nov. 20, 2023
(86) PCT No.: PCT/KR2023/018684

§ 371 Date: May 15, 2025

(30) Foreign Application Priority Data
Nov. 21, 2022 (KR) 10-2022-0156032
Aug. 31, 2023 (KR) 10-2023-0115490

(51) Int. Cl. A61K 36/31; A61K 31/194; A61K 31/198; A61K 35/745; A61K 35/747; A61P 25/32

(52) U.S. Cl. CPC A61K 36/31; A61K 31/194; A61K 31/198; A61K 35/745; A61K 35/747; A61P 25/32

(57) ABSTRACT

The present invention relates to a composition for hangover relief and liver function improvement, comprising L-ornithine, and a preparation method therefor. According to the present invention, the advantages are achieved of having low burden on the human body, while at the same time exhibiting a sufficient hangover relief effect. Accordingly, the embodiments of the present invention provide hangover relief and liver function improving effects.

### TECHNICAL FIELD

The following exemplary embodiments relate to a composition for hangover relief and liver function improvement, including a raw powder material, and a preparation method therefor.

### BACKGROUND ART

About 10% of ethanol absorbed by drinking is excreted from the body through respiration or urine, and the remaining 90% thereof is subjected to metabolism in the digestive organs or liver. Alcohol metabolism occurs in the liver, and ethanol is oxidized to acetaldehyde through alcohol dehydrogenase (ADH), and the acetaldehyde is oxidized to acetic acid through aldehydedehydrogenase (ALDH). The oxidation process requires nicotinamide adenine dinucleotide (NAD). The produced acetic acid is converted into carbonic acid gas and water through acetyl coenzyme A, and carbonic acid gas is discharged from the lungs and water is discharged out of the body through urine, etc. Excessive intake of alcohol causes a hangover phenomenon, and the hangover accumulates alcohol and acetaldehyde in the human body, which is known to act as a major toxic substance in each organ, thus having negative effects on the human body, such as headache, fatigue, light and sound sensitivity, muscle pain, eye redness, thirst, nausea, vomiting, stomach pain and the like.

In order to relieve such a hangover, as in Korean Registered Patent Publication No. 10-0853078, a number of functional beverages or compositions, particularly functional beverages, etc., containing ingredients extracted from natural materials, have been developed, and these functional beverages are consumed alone after drinking or through a method of adding the same to alcoholic beverages with a high alcohol content before drinking. However, the effect of relieving hangovers is still not so significant. In addition, drinks containing some herbal medicines may cause abdominal bloating, abdominal pain, etc., in some cases, thus arousing a controversy over whether those drinks are harmful to the human body or not. Accordingly, it is necessary to develop a composition which exhibits a sufficient hangover relief effect while imposing a low burden on the human body at the same time.

### RELATED ART REFERENCE

### Patent Documents

(Patent Document 1) Korean Registered Patent Publication No. 10-0853078

### DISCLOSURE

### Technical Problem

The following exemplary embodiments have been devised to solve the above-mentioned problems, and an object of the present invention may be to provide a composition exhibiting a sufficient hangover relief effect while imposing a low burden on the human body, and a preparation method therefor.

### Technical Solution

To achieve the object of the present invention as described above, one embodiment of the present invention may provide a food composition for hangover relief and liver function improvement, including: L-ornithine, potassium citrate, cabbage powder, and one or more lactic acid bacteria selected from the group consisting of bifidobacteria and lactobacillus, and a preparation method therefor.

One embodiment of the present invention may provide a method for preparing a food composition for hangover relief and liver function improvement, the method including: extracting and drying cabbage to prepare cabbage powder; and mixing the cabbage powder with L-ornithine, potassium citrate, and one or more lactic acid bacteria selected from the group consisting of bifidobacteria and lactobacillus.

### Advantageous Effects

According to the present invention, there are advantages of exhibiting a sufficient hangover relief effect while imposing a low burden on the human body at the same time. Accordingly, the exemplary embodiments of the present invention can provide hangover relief and liver function improving effects.

### MODE FOR INVENTION

Hereinafter, exemplary embodiments will be described in detail with reference to the accompanying drawings. However, various changes may be made to the exemplary embodiments, and thus the scope of the patent application is not limited to or restricted by these exemplary embodiments. It should be understood that all changes, equivalents, or substitutes to the exemplary embodiments are included in the scope of the rights.

Specific structural or functional descriptions of the exemplary embodiments are disclosed for the purpose of illustration only, and may be modified and implemented in various forms. Accordingly, the exemplary embodiments are not limited to a specific disclosure form, and the scope of the present specification includes changes, equivalents, or substitutes included in the technical spirit.

Terms such as “first,” “second” or the like may be used to describe various components, but these terms should be interpreted only for the purpose of distinguishing one component from another component. For example, a first component may be referred to as a second component, and similarly, the second component may also be referred to as the first component.

It will be understood that when an element is referred to as being “connected” to another element, the element may be directly connected to or linked to the other element, or intervening elements may be present therebetween.

The terms used in the exemplary embodiments are used only for the purpose of description and should not be construed as limiting. The terms of a singular form may include plural forms unless otherwise specified. In the present specification, the terms “comprise,” “have,” or the like are intended to designate that the features, the numbers, the steps, the operations, the components, the parts or combinations thereof are present, and are not to be understood as excluding the possibility that one or more other features, numbers, steps, operations, components, parts or combinations thereof may be present or added.

All the terms used herein including technical or scientific terms have the same meaning as commonly understood by those ordinary skilled in the art, to which the exemplary embodiments pertain, unless defined otherwise. Such terms as those defined in a generally used dictionary are to be interpreted to have the meanings equal to the contextual meanings in the relevant art, and are not to be interpreted to have ideal or excessively formal meanings, unless clearly defined in the present application.

The features and advantages of the present invention as well as methods for achieving them will be apparent with reference to exemplary embodiments described in detail hereinafter along with the accompanying drawings. However, the present invention is not limited to the exemplary embodiments disclosed hereinafter, but will be implemented in various different forms. Hereinafter, the following exemplary embodiments will be suggested for better understanding of the present invention and are provided only for the purpose of completely illustrating the scope of the present invention to those skilled in the art, and thus the present invention will be defined only by the scope of the claims thereto.

In exemplary embodiments of the present invention, all the terms used herein including technical or scientific terms have the same meaning as commonly understood by those ordinary skilled in the art, to which the present invention pertains, unless defined otherwise. Such terms as those defined in a generally used dictionary are to be interpreted to have the meanings equal to the contextual meanings in the relevant art, and are not to be interpreted to have ideal or excessively formal meanings, unless clearly defined in the exemplary embodiments of the present invention.

The shapes, sizes, ratios, angles, numbers, and the like disclosed in the accompanying drawings for describing the exemplary embodiments of the present invention are for illustration only, and the present invention is not limited thereto. In addition, in the description of the present invention, a detailed description of known arts incorporated herein will be omitted when it may make the subject matter of the present invention unnecessarily unclear. When “include,” “have,” “consist of” and the like mentioned in the present specification are used, other parts may be added unless “only” is used. In the case of expressing the components in the singular, it includes the case of including the same in the plural unless otherwise specified.

In interpreting the component, it is interpreted as including an error range even if there is no separate explicit description.

The size and thickness of each component shown in the drawings are illustrated for convenience of description, and the present invention is not necessarily limited to the size and thickness of the illustrated component.

Each feature of the various exemplary embodiments of the present invention may be partially or entirely connected or combined with each other, technically various interlocks and operations may be possible, as fully understood by those skilled in the art, and each exemplary embodiment may be implemented independently of each other or may be implemented together in an associative relationship.

Hereinafter, the present invention will be described in more detail with reference to the exemplary embodiments and drawings. However, it may be obvious that the present invention is not limited to the following exemplary embodiments and drawings.

### Hangover Relief and Liver Function Improvement

Conventional hangover relievers may be composed mainly of substances which help the metabolism of the liver. However, substances which respond to chronic reactions caused by drinking may have a problem in which the effect is not often dramatic, but small during an acute phase. Accordingly, the present inventors have found it necessary to analyze acute phase reactions of the liver, the gastrointestinal tract, and blood, which occur during heavy drinking, so as to remove the main factors which cause hangovers from a medical point of view, and have completed the present invention with the aim of minimizing the effects of alcohol, which is absorbed in the order of gastrointestinal tract-liver-blood. More specifically, the present invention has been completed with the aim of 1) enhancing alcohol decomposition in the gastrointestinal tract, 2) inhibiting alcohol absorption in the gastrointestinal tract, 3) improving the efficiency of alcohol metabolism in the liver and inhibiting hangover-causing substances, and 4) minimizing the systemic influence of alcohol in the blood, in the process of absorption inhibition-metabolism (decomposition) promotion-systemic symptom minimization-excretion promotion.

About 10% of ethanol (officially named ethyl alcohol) absorbed by drinking may be excreted from the body through respiration or urine, and 20% of the remaining 90% may be absorbed in the stomach and 80% thereof may be absorbed in the small intestines. Alcohol absorbed in the gastrointestinal mucosa may not require a special receptor and thus may be very quickly absorbed into the liver through the blood. It may be exposed to hepatocytes, subjected to metabolism, and absorbed into the central artery while being mixed with the blood of the portal vein venules and hepatic arterioles. The alcohol passed through this route may be oxidized in hepatocytes and converted into non-toxic acetate, which is used as energy (about 7.1 kcal for 1 g of alcohol). Since the metabolism of hepatocytes is limited in speed, the remaining alcohol, which has not been metabolized, may flow into the blood and lead to an increase in blood alcohol concentration. The excess alcohol, which is not oxidized, may flow into the blood, circulate throughout the body, and be metabolized again in the liver. Thus, excessive drinking may increase the blood alcohol concentration. Alcohol may easily pass through the cell membrane and mix well with the cellular lipid membrane, and thus interfere with the normal signaling of cells. In addition, alcohol may spread through the systemic circulation to various organs and appears as a systemic symptom. The absorbed alcohol may be metabolized to acetaldehyde in at least 90% of the liver and may be treated at a rate of one glass per hour.

A primary metabolism process in alcohol metabolism may be mainly subjected to 1) an alcohol dehydrogenase (ADH) system, 2) a microsomal ethanol oxidizing system (MEOS), and 3) a catalase system. Among them, clinically important pathways may be 1) and 2). The pathway 1) may be a main pathway for alcohol decomposition, and correspond to a pathway which is oxidized to acetaldehyde by ADH enzyme present in the cytoplasm of hepatocytes. Acetaldehyde, an intermediate substance, may be a very unstable and toxic substance, and may be one of the main factors of liver damage (causing hangovers such as facial redness, tachycardia, headache, and vomiting). The acetaldehyde may be rapidly converted into acetic acid by aldehydedehydrogenase (ALDH) present in the mitochondria (secondary metabolism process). The metabolism of acetaldehyde in mitochondria may rapidly occur, but ALDH mutations may slowly appear in many Asians, thus leading to the accumulation of acetaldehyde, which is considered a major factor for hangovers. Increasing acetaldehyde concentration may lead to a variety of hangover symptoms and inflammatory reactions. Above pathway 2) may account for 10 to 20% of alcohol metabolism and may be a pathway for metabolizing various drugs. Above pathway 2) may occur when the ADH pathway is not sufficient for alcohol metabolism due to heavy drinking, and ethanol may be oxidized to acetaldehyde. As drinking is repeated for a long period of time, the enzyme capacity may be up-regulated by 5 to 10 times, which makes a drinker feel that drinking capacity has increased. More specifically, two electrons in NADPH may be transferred to CYP2E1 (Cytochrome P450), a membrane protein located in the hepatocyte endoplasmic reticulum membrane, and ethanol may be attached to CYP2E1 and oxidized to acetaldehyde. In this process, reactive oxygen species (ROS) may be generated. The ROS may be converted into hydrogen peroxide (H₂O₂) to attack the cell membrane (lipid) and may suppress the electron transport system and oxidative phosphorylation processes. Accordingly, cells may undergo inflammatory reactions and die, and an exposure to the ROS in the long run may cause diseases such as cirrhosis. In a secondary metabolism process during alcohol metabolism, if a large amount of acetaldehyde, an intermediate substance, is accumulated, it may not be converted to acetic acid at a certain rate or more, and thus may flow into the blood through diffusion and show systemic symptoms (occurrence of hangover symptoms such as vomiting, redness, and nausea). Acetaldehyde may be metabolized through ALDH in the mitochondria to acetate, which is the final oxide.

In the primary/secondary metabolism process, the reduced product NADH may increase, thus increasing the ratio of NADH/NAD⁺. The increase in NADH may cause metabolic confusion. In the general aerobic metabolism process, carbohydrates/fats/proteins may be metabolized in the TCA cycle to produce NADH, which may be converted to ATP to generate energy. When NADH is increased due to drinking, the general aerobic metabolism process may be inhibited on the contrary. Since NADH is sufficient, metabolism processes (glycolysis/gluconeogenesis/TCA cycle (energy generation)/fatty acid oxidation may proceed in a direction in which energy generation is suppressed. Thus, energy generation may be suppressed and hypoglycemia may occur. As a result of alcohol metabolism, acetic acid may accumulate, thus resulting in fat accumulation and lactic acid production, which may accumulate fatigue. Fasting blood sugar may fall or remain lower than usual, and postprandial blood sugar may rise.

In addition, drinking may cause a diuretic action, thus leading to potassium deficiency, and as a result, the response of cells to ROS may decrease and the decrease in ATP production may be intensified. As for potassium depletion, alcohol may interfere with the secretion of antidiuretic hormones produced in the posterior pituitary and may exacerbate hyponatremia. For example, potassium loss may stimulate ADH activity, thus increasing the amount of body fluid which is resorbed, and consequently reducing the sodium concentration in the body. Potassium loss may also increase thirst through hormone mechanisms, thereby increasing water intake. When potassium in extracellular fluid (ECF) is insufficient, potassium may be transferred from intracellular fluid (ICF) to the extracellular fluid due to a difference in concentration. Accordingly, potassium in the cytoplasm may be reduced. In the meantime, the inner membrane of mitochondria may not transmit positive ions well, but some amount of potassium may flow into the mitochondrial matrix, which may depend on the concentration difference.

With the introduction of potassium, the mitochondria may maintain the volume to keep the membrane taut and maintain a potential difference. When potassium in the cytoplasm is reduced, K⁺ outflow toward the mitochondrial matrix may decrease, thus resulting in a shortage of potassium in the mitochondria. This may tend to result in a general reduction in mitochondrial functions, including energy production. In this way, a situation in which ATP synthesis is poor and cell respiration is poor may become a condition in which ROS is easily formed. In an over-reduced state with a large amount of NADH/NAD⁺, excess electrons may be likely to be transferred to oxygen and combined, thus resulting in a high probability of ROS being generated. In addition, a large amount of ROS may be also produced in the alcohol decomposition process through the microsomal ethanol oxidizing system (MEOS) due to excessive drinking. ROS may cause inflammation, aging, cancer, degenerative diseases, etc. Under the stress conditions of having ROS produced, the mitochondria may activate the ATP-dependent-potassium channel, thus allowing potassium to flow into the mitochondrial matrix. As a result, the membrane potential difference may be lowered and superoxide anions may be less generated to inhibit the production of ROS. Thus, maintaining the potassium concentration of the cytoplasm may be expected to inhibit the production of ROS, thereby inhibiting inflammatory reactions and normalizing mitochondrial functions. Since acetaldehyde is decomposed through ALDH in the mitochondria, it may be expected to become active as well. The supplementation of potassium may be expected to inhibit both inflammatory reactions and acetaldehyde production, which are cited as the main causes of hangover.

Accordingly, the present inventors have found that the direct cause of hangover may be blood cytokines rather than alcohol and acetaldehyde, considering that 1) the blood ethanol concentration reaches a maximum in 60 to 90 minutes after drinking and decreases over time, 2) the blood acetaldehyde concentration reaches a maximum within one hour after drinking, and 3) the hangover reaches a maximum in 12 to 14 hours after drinking (in this case, alcohol and acetaldehyde are almost fully metabolized). In particular, the present inventors have found that an expression time of cytokines after alcohol stimulation may correspond to an expression time of hangover, and that when cytokines are injected into healthy people, hangover-like symptoms such as gastrointestinal disorder, headache, chill, fatigue, and vomiting may be expressed. Accordingly, the present inventors have hypothesized that the main factor of hangover may be cellular immunity decline mediated by IL-10 and 12 (IL-10, IL-12).

### Fatigue Recovery

In one aspect, the present invention may provide a food composition for fatigue recovery. Ammonia may reflect a state of fatigue well during exercise. The central fatigue mechanism of ammonia may act in such a way that when the large accumulation of toxic ammonia leads to a disorder in motor functions such as lethargy, convulsion, ataxia, coma, etc., and the peripheral fatigue mechanism may act in such a way that the accumulation of ammonia in the muscles stimulates the afferent nerves associated with the detection of muscle pains, thereby causing fatigue. The composition according to the present invention may reduce the concentration of ammonia, cortisol, and lactic acid, which are blood fatigue materials, and may rapidly increase the concentration (pH) of hydrogen ions, thereby exhibiting an effect of rapidly ameliorating fatigue after exercise.

### Food Composition for Hangover Relief and Liver Function Improvement

In one aspect, the present invention may provide a food composition for hangover relief and liver function improvement, including: L-ornithine, potassium citrate, cabbage powder, and one or more lactic acid bacteria selected from the group consisting of bifidobacteria and lactobacillus.

In particular, the present inventors have found that the ingredients included in the composition as active ingredients may exhibit a sufficient hangover relief effect while imposing a low burden on the human body, thereby completing the present invention, and thus have found a method of approaching hangover relief from a medical point of view by applying a safe substance licensed as a food. The composition of the present invention may be utilized for hangover relief and liver function improvement.

In the present invention, the term “extract” may refer to an active ingredient isolated from a natural product, and may be obtained by an extraction process using water, an organic solvent, or a mixed solvent thereof, and may include an extracted liquid, a dry powder thereof, or all forms formulated by using the same. The extracted liquid may be used directly or may be used as a “powder” after concentration and/or drying. When the extraction is performed using an organic solvent, the organic solvent used herein may include methanol, ethanol, isopropanol, butanol, ethylene, acetone, hexane, ether, chloroform, ethyl acetate, butyl acetate, dichloromethane, N,N-dimethylformamide (DMF), dimethyl sulfoxide (DMSO), 1,3-butylene glycol, propylene glycol, or a mixed solvent thereof, and the extraction may be performed at room temperature or at a higher temperature under conditions in which the active ingredient is not destroyed or is minimized. Depending on the organic solvent used for extraction, the effective ingredients of the drug may differ in the degree of extraction and the degree of loss, and thus an appropriate organic solvent may need to be selected and used. An extraction method is not particularly limited, and may include, for example, hot water extraction, cold precipitation extraction, ultrasonic extraction, reflux extraction, and the like.

The solvent extract may further include filtering the extract to remove suspended solid particles. Particles may be filtered out by using cotton, nylon, etc., or ultrafiltration, freeze filtration, centrifugation, etc., may be used, but is not limited thereto. When the extracted liquid is concentrated, the method used herein may include concentration under reduced pressure, concentration under reverse osmosis, etc. The drying after concentration used herein may include freeze drying, vacuum drying, hot air drying, spray drying, reduced pressure drying, foam drying, high frequency drying, infrared drying, etc., but is not limited thereto.

According to the composition of the present invention, it may be provided that 1) the absorption of alcohol is inhibited, 2) the decomposition of alcohol and a by-product thereof is activated, and 3) the inflammatory reaction caused by alcohol and the by-product thereof is inhibited. More specifically, the composition of the present invention may exhibit the effects of inhibiting absorption in the gastrointestinal tract when an excessive amount of alcohol is drunk, promoting liver decomposition, inhibiting the generation of inflammatory substances, and optimally maintaining the function of the liver to facilitate metabolism. The composition of the present invention may be taken together with drinking or immediately after drinking, and thus may be particularly excellent in short-term effects of solving a phenomenon occurring in an acute phase.

The present inventors have named the composition of the present invention having hangover relief and liver function effects “Morning WOW.”

* The “active ingredient” may refer to a component exhibiting a desired activity or a component capable of exhibiting activity together with a carrier having no activity per se.

The “L-ornithine” may be a basic amino acid which plays an important role as an intermediate in the urea cycle. In the urea cycle, L-ornithine may flow into the mitochondria from the cytoplasm, capture an ammonia group from the mitochondria, synthesized into L-citrulline, and come out into the cytoplasm to make arginosuccinate. This may serve to remove a nitrogen group from the mitochondria. Further, the arginosuccinate may receive a second amino group of urea from aspartate, the carbon group of aspartate may be dropped into fumarate, and the nitrogen group may be transferred to arginine, thereby synthesizing harmless urea. The urea may be then excreted through the kidney. Representative two cycles which act in the body may include a citric acid (TCA) cycle which produces energy and a urea circuit which processes ammonia. The citric acid cycle may occur in the mitochondria, and the urea cycle may occur across the cytoplasm+mitochondria. The two circuits may interact with each other, for example, by sharing an intermediate metabolite (argininosuccinate shunt), using a portion of the ATP produced in the citric acid cycle (4ATP) in the urea cycle, and the like. Amino acid, which is a metabolite, may generate ammonia (nitrogen compound) as a toxic substance, which may be converted into harmless urea through the urea cycle and discharged through the kidney.

Under drinking conditions, alcohol decomposition by ADH may occur preferentially over mitochondrial metabolism of other nutrients, thus resulting in accumulation of NADH and a higher NADH/NAD⁺ ratio. This may slow down the TCA cycle which converts NAD⁺ to NADH. Oxaloacetate of the TCA cycle may receive an amino group of glutamate to make aspartate, and may transfer the amino group to the urea cycle via arginosuccinate. However, in the case of drinking, the NADH/NAD⁺ may be high, and thus aspartate may be metabolized by the pathway of producing oxaloacetate and lowering NADH. As a result, the amino group of aspartate may not be transferred to the urea cycle, but may be transferred to alpha-glutarate to generate glutamate. Thus, the removal of amino groups from hepatocytes may be reduced, and ammonium ions may be expected to accumulate, which may lead to abnormal mitochondrial functions. In the present invention, ornithine, which enters the mitochondria and collects nitrogen groups, has been supplemented.

The L-ornithine may directly enter the mitochondria, may capture nitrogen from ammonia, may be converted into citrulline, and may be discharged out of the mitochondria. In other words, it may play a role of extracting the same to the cytoplasm. In a situation in which ammonia removal is reduced due to an increase in blood alcohol concentration, the L-ornithine may exert a direct effect on ammonia removal. In addition, carbon dioxide required for carbamoyl phosphate produced in the process of delivering ammonium to ornithine may be supplied into the mitochondria and may be expected to maximize the effect of removing nitrogen groups with ornithine. The present invention has focused on normalizing the mitochondria by preventing amino groups from accumulating in the mitochondria. To this end, potassium citrate has been supplemented to supplement the carbon dioxide whose production is slowed due to the slowing of the TCA cycle. As the ammonia is effectively removed from the mitochondria, acetaldehyde decomposition in the mitochondria may be normalized, leading to a reduction in ROS production, thus making a virtuous cycle. Then, the mitochondria may be normalized, thus exhibiting the effect of eventually decomposing acetaldehyde from ALDH in the mitochondria.

The “potassium citrate” may be an alkaline organic salt compound and act as an alkalizing agent. Potassium citrate may have a buffering action against the gastric acidity, which is believed to appear by bicarbonate after being absorbed by chlorine ions before being absorbed. 90% of the potassium citrate may be ionized at physiological pH, and the ionized citric acid may act as a trivalent anion. Blood bicarbonate ions (HCO₃⁻) and hydrogen ions (H⁺) may be converted into water and carbon dioxide in the body and may be balanced (HCO₃⁻+H+<->H₂O+CO₂). Citric acid liberated in the blood may bind to H⁺ and exhibit the effect of liberating HCO₃⁻ in the blood, thus acting as an alkali. This may be expected to prevent lactic acidosis of blood caused by the generation of lactic acid after excessive drinking, thereby exhibiting an effect of preventing one of the causes of hangover. Potassium citrate may be rapidly absorbed through the gastrointestinal tract. Substances absorbed from the gastrointestinal tract may immediately flow into the liver and enter hepatocytes. As lactic acid are produced under excessive drinking, citric acid may be more ionized near acidic hepatocytes, capture H⁺, and release HCO₃⁻ and increase the blood pH to normalize the blood pH. In the meantime, CO₂ generated in this process may smoothly spread to the inside due to the nature of the inner membrane of the mitochondria through which gas permeates well. Potassium citrate may play a role in supplying carbon dioxide and potassium directly to hepatocytes. In order to convert a nitrogen group into urea in the urea cycle, NH₄⁺ and carbon dioxide may need to react in the mitochondria to form carbamoyl phosphate. Since the TCA cycle does not smoothly work during excessive drinking, CO₂ may be less generated, and thus it may be predicted that the smooth supply of external CO₂ is necessary for activation of the urea cycle. At this time, the citric acid of potassium citrate may react with H⁺ to make water and carbon dioxide. Carbon dioxide may flow into cells to temporarily form hypercarbia. Thus, carbon dioxide may smoothly pass through the inner membrane of the mitochondria to supply carbon dioxide necessary for forming carbamoyl phosphate. Thus, potassium citrate may activate a metabolic process in which L-ornithine captures an ammonia group in the mitochondria and is converted into citrulline. As a result, it may be expected to achieve the effect of preventing the accumulation of ammonia groups in hepatocytes and normalizing the mitochondria, thereby facilitating the removal of acetaldehyde.

Potassium may be highly excreted by drinking. Since potassium in the extracellular fluid is reduced, extracellular shift may occur, and as a result, potassium may flow into the cells through Na/K ATPase, and the cells may use ATP more. In order for hepatocytes to respond to ROS generated during the alcohol decomposition process, K⁺ may be required, but potassium may be excreted when alcohol is consumed, thus causing a situation where the same is rather insufficient.

Hepatocytes, which are more problematic in responding to ROS due to the lack of potassium, may be activated with the supplementation of potassium. The potassium citrate may be absorbed in the gastrointestinal tract faster than other potassium-containing agents, and thus may be absorbed in hours and reach hepatocytes fast, thereby achieving an effect of directly supplying potassium to the hepatocytes. The supply of potassium may reduce ROS generated in mitochondria, and thus may be expected to reduce inflammatory reactions which are caused by the ROS in the future. Thus, it may be expected to contribute to alleviating hangovers after drinking. In the meantime, due to the nature of citric acid reacting with H⁺ when the pH is low, it may be expected to increase the pH of the stomach by reacting with H⁺ in the stomach. During drinking, gastric acid may be excessively secreted, which may be expected to alleviate symptoms such as heartburn, nausea, vomiting, etc., immediately after drinking.

The present inventors have constructed the potassium citrate as an active ingredient for the purpose of directly introducing K⁺ to prevent K⁺ depletion of hepatocytes. In the meantime, citrate may also be expected to contribute to the activation of urea cycle along with the L-ornithine by supplying a carbon dioxide group. In particular, the potassium citrate may be absorbed faster through the gastrointestinal tract than other components, and thus may quickly reach hepatocytes, which may be very helpful in alcohol degradation.

The “cabbage powder” may be a component for protecting the stomach when drinking alcohol, and may be obtained by cutting cabbage, drying the same to remove moisture, and then grinding the same. The cabbage powder may be a natural anti-ulcer food and contain a component which protects and strengthens the gastric mucosa, may be effective in preventing cancer such as stomach cancer and colorectal cancer, and may be effective in diet, preventing constipation, and preventing skin aging due to abundant dietary fiber and low calorie content. The present inventors have constructed the cabbage powder as an active ingredient for the purpose of exerting an alcohol absorption inhibitory effect, an antioxidant/anti-inflammatory effect, and an acetaldehyde decomposition promoting effect of a dietary fiber, in addition to the effect of protecting the gastric mucosa. Furthermore, the sulforaphane contained in the cabbage powder may have an excellent antioxidant effect, a cell stress reduction effect, and an inflammation reduction effect when oxidative stress is induced to cells. Moreover, the sulforaphane may activate hsALDH, which is an isoform of ALDH contained in the salivary gland and is normally deactivated, so as to induce ALDH and decompose acetaldehyde, which may be helpful to people who have difficulty in decomposing acetaldehyde due to ALDH gene variation. The ALDH variation of Koreans may be estimated to be about 30% of the population. Accordingly, it may be effective to reduce the oxidative stress of hepatocytes due to ROS generated during excessive alcohol drinking and to reduce inflammation resulting from promoting acetaldehyde decomposition.

Alcohol may be transferred to the large intestine through the flow of blood, so that the blood alcohol concentration may become equal to the alcohol concentration in the large intestine. In other words, the maximum concentration may be also achieved in the large intestine between 30 minutes and 90 minutes after drinking, in which the blood alcohol concentration reaches a maximum. In the large intestine, alcohol-acetaldehyde metabolism may occur by ADH of E. coli, thus producing acetaldehyde. Since the flora of the large intestine has a very low activity of ALDH compared to ADH, the concentration of acetaldehyde in the large intestine may be the highest in the body. In general, it may be expected that ingested food takes nine hours or more to reach the large intestine, acetaldehyde is produced before lactobacillus is delivered to the large intestine along with the food, and lactobacillus ingested before drinking will not have a significant effect on the removal of acetaldehyde on the day of drinking. Accordingly, as a dietary fiber, it may be useful to use a cabbage including a large amount of insoluble dietary fiber, which may activate the general intestinal motion to increase the speed of the intestinal tract, increase a defecation rate, reduce the absorption of acetaldehyde into the blood, and form the bulk, thereby promoting the intestinal motion. In particular, the heat-treated cabbage powder may be effective in treating hepatitis inflammation by reducing the expression of inflammatory mediators iNOS and COX-2 and the pro-inflammatory cytokine IL-1β, which are involved in the acute inflammatory response accompanying liver damage. The cabbage powder used in the present invention may be used by heat treatment at about 121° C. In addition, s-methylmethionine sulfonium chloride (MMSC), which is a typical antipeptic ulcer in cabbage, may promote the production of prostaglandins, which are local hormones secreted from the gastric mucosa, and prostaglandins secreted into the gastric cavity may impart gastric mucosal defense ability from gastric acids or other attack factors.

As described above, the cabbage powder used in the present invention may not be a juice obtained from lyophilized cabbage, but may be a heat-treated cabbage powder containing an insoluble dietary fiber. Accordingly, it may exert the effect of the insoluble dietary fiber which prevents constipation and enteritis by absorbing water to increase feces and shortening the time taken to pass through the intestine, in addition to the effect of cabbage preventing acute gastritis. In addition, the lactic acid bacteria used in the present patent material may be mixed with the dietary fiber, and thus an initial alcohol decomposition caused by ADH of the lactic acid bacteria may also be expected. Thus, it may have an anti-inflammatory effect in the stomach, may absorb alcohol from the small intestine which is a main place for absorbing alcohol, so as to lower an alcohol absorption rate, and may be expected to have the lactobacillus mixed with cabbage break down alcohol, along with the effect of promoting excretion. In addition, since there is an effect of reducing ROS generated in the liver and promoting acetaldehyde decomposition to reduce the inflammatory reactions, it may be expected to be useful for alleviating hangovers.

The “bifidobacteria” and “lactobacillus” may have the ability for almost all strains to decompose alcohol and/or acetaldehyde. Thus, when administered together with cabbage, which is a dietary fiber, it may be expected to decompose the alcohol absorbed by the cabbage, thereby accomplishing an alcohol decomposition effect at the beginning of drinking.

The “bifidobacteria” may be a gram-positive genus, non-mobile, and often classified as anaerobic bacteria. They may be found in the gastrointestinal tract, vagina, and mouth of mammals. Bifidobacteria may be one of the major genera of bacteria which make up the colon flora in mammals. Bifidobacterial organisms may be commercially available and sold as probiotics for human consumption. For example, Complete Probiotics capsule by Dr. Mercola® may contain 70 billion organisms including three species of bifidobacteria. Other commercial sources of bifidobacteria may be also commercially available (e.g., Align® Bifantis® Bifidobacterium infantis 35624). Any species of bifidobacteria which may be used in the present invention may be suitable, because strains of this genus are known to live and function in human digestive organs and are known to be safe for human consumption. Such systems used in the methods and compositions of the present invention may include, without limitation, B. infantis, B. breve, B. adolescentis, B. animalis, B. pseudolongum, B. thermophilum, B. indicum, B. asteroids, B. lactis, B. longum, B. coagulans, B. dentium, B. infantis, B. animalis ssp. lactis, and B. bifidum.

The “lactobacillus” may be a gram-positive, facultative anaerobic or microaerobic rod-shaped bacterium. This genus may constitute a major part of the bacterial (lactobacilli) family which converts lactose and other sugars into lactic acids. They may be found in the human gastrointestinal tract and vagina. Some strains of lactobacillus may have potential therapeutic properties, including anti-inflammatory and anti-cancer abilities. In addition, the lactobacillus may promote gastric ulcer treatment. There may be various commercial sources of lactobacillus (e.g., Health & Wellness 30-Capsules or Digestive Health 30-Capsules sold by Culturelle®, Cromwell, Connecticut; Nutrition Now® Pro-Biotics Acidophilus from GNC; FoodScience® of Vermont Lactobacillus Acidophilus capsules). Any species of lactobacillus which may be used in the present invention may be suitable, because strains of this genus are known to live and function in human digestive organs and are known to be safe for human consumption. Such systems used in the methods and compositions of the present invention may include, without limitation, L. acidophilus (e.g., L. acidophilus DDS-1, L. acidophilus LA-5, L. acidophilus NCFM), L. bulgaricus, L. jugurti, L. helveticus, L. salivarius, L. casei, L. plantarum, L. salivarius, L. rhamnosus (e.g., L. rhamnosus A), L. paracasei, L. lactis, L. infantis, and L. brevis.

The “bifidobacteria” and “lactobacillus” may be representative genera used as probiotic lactic acid bacteria. Since the strength of acidity, the time of exposure to acid, and the type of strain are the main factors affecting the survival of lactic acid bacteria in the stomach, among them, the “bifidobacteria” and the “lactobacillus” may survive in an acidic environment, and thus may be widely used in lactic acid bacteria products. In particular, L. acidophilus and B. longum may survive better in the stomach and adhere better to the gastric mucosa.

Alcohol may be mainly absorbed in the small intestine and cause barrier damage, and lactic acid bacteria may show the effect of ameliorating barrier damage and may reduce endotoxin absorption to reduce liver damage. Oxidative stress caused in the intestine by alcohol may hinder the tight binding of intestinal epithelial cells, thus increasing intestinal permeability. Acetaldehyde may also disrupt microtubules in intestinal epithelial cells, thus increasing intestinal permeability. When intestinal permeability increases, endotoxin present in the cell wall of gram-negative bacteria present in the intestine may flow into the bloodstream, which may promote the secretion of inflammatory cytokines such as TNF-α, IL-1, and IL-6, thus causing liver cell damage (https:/koreascience.kr/article/JAKO201409649928775.pdf). This inflammatory cytokine may be also found in alcohol metabolism, and it may be predicted that increased intestinal permeability due to drinking will result in increased liver damage. Thus, lactic acid produced by lactic acid bacteria may proliferate intestinal stem cells to regenerate intestinal mucosal epithelial cells. “Bifidobacteria” and “lactobacillus” used as the materials of the present patent may have excellent ability to withstand gastric acid, and thus may produce lactic acid in the small intestine and proliferate intestinal stem cells, which may be expected to recover the intestinal mucosa damaged by drinking. Thus, they may be expected to contribute to reducing liver damage caused by excessive drinking and to relieve hangovers. Since it takes time for food to reach the large intestine, acetaldehyde occurs immediately after drinking in the large intestine and reaches the maximum concentration before food reaches the large intestine, and the degree of alcohol decomposition in the early stage of drinking determines the degree of hangover the next day, the lactic acid bacteria used in the present invention may include “lactobacillus” which is well resistant to gastric acid and “bifidobacteria” which is much present in the large intestine, focusing on the contribution of lactic acid bacteria to the recovery of small intestine damage rather than acetaldehyde decomposition, which mainly occurs in the late stage of drinking. Accordingly, the combination of the “bifidobacteria” and “lactobacillus” lactic acid bacteria may exhibit an effect of reducing the irritation to the stomach caused by alcohol and alleviating erosive gastritis, heartburn, and nausea at the same time. Furthermore, the lactic acid bacteria may be promoted to proliferate in the presence of dietary fiber, thereby exhibiting a synergistic effect such as a gastric ulcer healing effect, etc., with cabbage powder.

In one embodiment, with regard to the food composition, the L-ornithine may be contained in an amount of 15 to 30 wt % based on the total weight of the composition, the potassium citrate may be contained in an amount of 2 to 12 wt % based on the total weight of the composition, the cabbage powder may be contained in an amount of 30 to 50 wt % based on the total weight of the composition, and the lactic acid bacteria may be contained in an amount of 2 to 14 wt % based on the total weight of the composition.

More specifically, the L-ornithine may be contained in an amount of 15 wt % or more, 16 wt % or more, 17 wt % or more, 18 wt % or more, 19 wt % or more, 20 wt % or more, 21 wt % or more, 22 wt % or more, 23 wt % or more; 30 wt % or less, 29 wt % or less, 28 wt % or less, 27 wt % or less, 26 wt % or less, 25 wt % or less, 24 wt % or less, and 23 wt % or less based on the total weight of the composition.

In one embodiment, the potassium citrate may be contained in an amount of 2 to 12 wt % based on the total weight of the composition. More specifically, the potassium citrate may be contained in an amount of 2 wt % or more, 3 wt % or more, 4 wt % or more, 5 wt % or more, 6 wt % or more, 6.7 wt % or more; 12 wt % or less, 11 wt % or less, 10 wt % or less, 9 wt % or less, 8 wt % or less, 7 wt % or less, and 6.7 wt % or less based on the total weight of the composition.

More specifically, the cabbage powder may be contained in an amount of 30 wt % or more, 31 wt % or more, 32 wt % or more, 33 wt % or more, 34 wt % or more, 35 wt % or more, 36 wt % or more, 37 wt % or more, 38 wt % or more, 39 wt % or more, 40 wt % or more; 50 wt % or less, 49 wt % or less, 48 wt % or less, 47 wt % or less, 46 wt % or less, 45 wt % or less, 44 wt % or less, 43 wt % or less, 42 wt % or less, 41 wt % or less, and 40 wt % or less based on the total weight of the composition.

More specifically, the lactic acid bacteria may be contained in an amount of 2 wt % or more, 3 wt % or more, 4 wt % or more, 5 wt % or more, 6 wt % or more, 7 wt % or more; 14 wt % or less, 13 wt % or less, 12 wt % or less, 11 wt % or less, 10 wt % or less, 9 wt % or less, 8 wt % or less, 7 wt % or less, and 6 wt % or less based on the total weight of the composition.

In one embodiment, with regard to the food composition, the L-ornithine, potassium citrate, cabbage powder, and lactic acid may be contained in a ratio of 2 to 5:1 to 2:4 to 10:1 to 2, in a ratio of 3 to 4:1 to 2:5 to 8:1 to 2, or in a ratio of 3 to 4:1 to 2:5 to 7:1 to 2 based on the weight.

In one embodiment, the food composition may further include Oenanthe javanica powder.

The “Oenanthe javanica powder” may contain persicarin which activates alcohol metabolism-related enzymes. Oenanthe javanica may be a perennial herb of the Umbeliferae family, naturally grow throughout Korea, and widely distributed and cultivated for food in areas ranging from the subpolar zone to the tropical zone, such as China, Japan, etc.

The Oenanthe javanica powder may be contained in an amount of 3 to 13 wt % based on the total weight of the composition. More specifically, the Oenanthe javanica powder may be contained in an amount of 3 wt % or more, 4 wt % or more, 5 wt % or more, 6 wt % or more, 7 wt % or more, 8 wt % or more, 8.14 wt % or more; 13 wt % or less, 12 wt % or less, 11 wt % or less, 10 wt % or less, 9 wt % or less, and 8.14 wt % or less based on the total weight of the composition.

In one embodiment, the food composition may further include Curcuma longa L extract.

The “Curcuma longa L extract” is known to have a pharmacological action such as a bile secretion promotion action, a uterine excitation action, a blood pressure lowering action, an analgesic action, etc. It has an effect of inhibiting the development of Staphylococcus aureus, fungi, etc., and has been proven to reduce hyperlipidemia (Korean Compendium of Materia Medica, authored by Ahn deok-gyun). Curcuma longa L may be is a root stem of the perennial grass. Curcuma longa L may be mainly grown in subtropical regions with heavy rain, ranging from southern China to Southeast Asia. It may also wildly grow in the forest areas of South Asia and Southeast Asia. Curcuma longa L may contain curcumin and essential oil components such as turmerone, zingerene, phellandrene, cineole, sabinene, bornol, dehydroturmerone, etc. The curcumin may be a kind of polyphenol and may exert the effect of protecting the liver and inhibiting systemic inflammatory reactions through anti-inflammatory and antioxidant effects. More specifically, it may change NF-κB signals to be involved in inflammatory cytokines (interleukin, phospholipase A2 production inhibition COX2, COX5), thereby exerting an anti-inflammatory effect.

The Curcuma longa L extract may be contained in an amount of 3 to 13 wt % based on the total weight of the composition. More specifically, the Curcuma longa L extract may be contained in an amount of 3 wt % or more, 4 wt % or more, 5 wt % or more, 6 wt % or more, 7 wt % or more, 8 wt % or more; 13 wt % or less, 12 wt % or less, 11 wt % or less, 10 wt % or less, 9 wt % or less, and 8 wt % or less based on the total weight of the composition.

According to one embodiment of the present disclosure, the composition may be a health functional food composition. In one embodiment, the food composition may be included to prepare a health functional food in any one form of powder, granule, pill, tablet, capsule, or beverage. A preparation method used herein may include a baking method and a far-infrared ray method, but is not limited thereto.

The term “health functional food” may refer to a food which uses a specific component as a raw material for the purpose of health assistance or a food produced and processed by extracting, concentrating, purifying, mixing, etc., a specific component contained in a raw material, and may refer to a food designed and processed to sufficiently exert a biological control function, such as a biological defense, on a living body by using the component, which may perform a function related to the disease prevention, amelioration, health recovery or the like.

There is no particular limitation on the type of health functional food in which the extracts according to the present disclosure may be used. For example, there may be ramen, other noodles, beverages, tea, drinks, alcoholic beverages, various soups, meat, sausage, bread, chocolate, candies, confectionery, pizza, gum, dairy products including ice cream, vitamin complexes, or the like. In addition, the health functional food may include a powder, granule, tablet, capsule or beverage form, and preferably include a beverage form. In addition, when the extracts according to the present disclosure are used as health functional foods, they may be added as they are or used together with other foods or food ingredients, which may be selected and appropriately used as necessary. Furthermore, in addition to the extracts according to the present disclosure, it may be possible to mix known additives with other suitable auxiliary ingredients which may be commonly contained in health functional foods according to the choice of those skilled in the art.

### Method for Preparing Food Composition for Hangover Relief and Liver Function Improvement

In another aspect, the present invention may provide a method for preparing a food composition for hangover relief and liver function improvement, the method including: preparing a mixture by mixing a powder raw material including L-ornithine, potassium citrate, cabbage powder, and one or more lactic acid bacteria selected from the group consisting of bifidobacteria and lactobacillus with water; making the mixture into pill to prepare a pill precursor; molding the pill precursor to smooth a surface thereof; and drying the pill precursor having a smoothly molded surface to prepare pill.

In another aspect, the present invention may provide a method for preparing a food composition for hangover relief and liver function improvement, the method including: extracting and drying cabbage to prepare cabbage powder; and mixing the cabbage powder with L-ornithine, potassium citrate, and one or more lactic acid bacteria selected from the group consisting of bifidobacteria and lactobacillus.

In the preparation method according to one embodiment of the present invention, the extraction may be performed by grinding the freeze-dried raw material, adding distilled water thereto, and then extracting at 75˜85° C. for two to four hours, and preferably adding distilled water to the freeze-dried raw material and repeatedly extracting at 80° C. three times for three hours, respectively.

According to one embodiment of the present invention, the preparing of the powder may be performed before the mixing, may be performed after the mixing, or may be performed simultaneously.

In one embodiment, the powder raw material and water may be mixed in a weight ratio of 1 to 3:1, and more specifically in a weight ratio of 7:3.

In one embodiment, a diameter of the pill precursor may be 4 to 6 mm, and more specifically 5 mm.

In one embodiment, packaging the pill in a square or rectangular four-sided packaging container may be further included.

In one embodiment, the drying may be performed at 35° C. to 37° C. for 60 to 72 hours.

In one embodiment, the preparation method may include the steps of: mixing each raw powder material; putting the mixed raw material and water in a kneader and kneading the mixture; putting the dough in a pill-making machine and making the mixture into a pill shape; putting the prepared pill into a molding machine and forming a pill surface; putting the formed pill into a dryer and drying the same; putting the dried pill in a pill sorter and sorting out defective pills; internally packing the sorted pill in a four-sided packing paper; moving the internally packed semi-finished product into a packing chamber and packaging the same into a separate case or a box for shipping; and moving the packaged finished product to a waiting place for shipping.

### EXAMPLE

Hereinafter, the present disclosure will be described with reference to examples. However, the following examples are provided only for the purpose of helping overall understanding of the present invention, and the scope of the present disclosure is not limited thereto.

### <Preparation Example 1> Preparation of Food Composition for Hangover Relief and Liver Function Improvement

Food compositions for hangover relief and liver function improvement according to Examples 1 to 4 of the present invention were prepared according to the compositions as shown in Tables 1 and 2 below.

TABLE 1

| Raw material | Example 2 (unit: wt %) | Example 2 (unit: g) |
|---|---|---|
| L-ornithine | 23 | 1.035 |
| Potassium citrate | 6.7 | 0.3015 |
| Cabbage powder | 40 | 1.8 |
| B. longum | 1 | 0.045 |
| L. casei | Total 6 | Total 0.27 |
| L. plantarum | | |
| B. breve | | |
| L. acidophilus | | |
| L. rhamnosus | | |
| B. animalis ssp. Lactis | | |
| L. lactis | | |
| Other components | Residue | Residue |

TABLE 2

| Raw material | Example 1 (unit: wt %) | Example 1 (unit: g) |
|---|---|---|
| L-ornithine | 23 | 1.035 |
| Potassium citrate | 6.7 | 0.3015 |
| Cabbage powder | 35 | 1.575 |
| B. longum | 1 | 0.045 |
| L. casei | Total 6 | Total 0.27 |
| L. plantarum | | |
| B. breve | | |
| L. acidophilus | | |
| L. rhamnosus | | |
| B. animalis ssp. Lactis | | |
| L. lactis | | |
| Oenanthe javanica | 8.14 | 0.3663 |
| powder | | |
| Curcuma longa L extract | 8 | 0.36 |
| Other components | Residue | Residue |

TABLE 3

| Raw material (unit: wt %) | Example 3 |
|---|---|
| L-ornithine | 23 |
| Potassium citrate | 6.7 |
| Cabbage powder | 40 |
| B. longum | 1 |
| L. casei | Total 6 |
| L. plantarum | |
| B. breve | |
| L. acidophilus | |
| L. rhamnosus | |
| B. animalis ssp. Lactis | |
| L. lactis | |
| Other components | Residue |

TABLE 4

| Raw material (unit: wt %) | Example 4 |
|---|---|
| L-ornithine | 30 |
| Potassium citrate | 8.73 |
| Cabbage powder | 52.15 |
| B. longum | 1.3 |
| L. casei | Total 7.82 |
| L. plantarum | |
| B. breve | |
| L. acidophilus | |
| L. rhamnosus | |
| B. animalis ssp. Lactis | |
| L. lactis | |
| Other components | Residue |

### <Reference Example 1> Preparation of Composition According to Comparative Example

Compositions according to each of Comparative Examples 1 to 4 were prepared in the same manner as in Example 1, respectively, except that L-ornithine was not included, potassium citrate was not included, cabbage powder was not included, and lactic acid bacteria were not included.

### <Experimental Example 1> Evaluation of Inhibition of Increasing Alcohol Concentration in Body

Ten male and female subjects were 30 to 40 years old and weighed 65 to 75 kg, and alcohol intake was 400 ml (alcohol content 19.5%). An experiment was conducted in such a way that an initial alcohol concentration was measured in 30 minutes after drinking, 5 g of the health functional food prepared in each of Examples and Comparative Examples was ingested, and an alcohol concentration (unit: mg/L) was measured with a breathalyzer in 90 minutes after drinking. A difference in alcohol concentration in 30 and 90 minutes after drinking is shown in Table 5.

TABLE 5

| | | | Comparative | Comparative | Comparative | Comparative |
|---|---|---|---|---|---|---|
| Classification | Example 1 | Example 2 | Example 1 | Example 2 | Example 3 | Example 4 |
| 1 | −0.05 | −0.08 | 0.05 | 0.08 | 0.03 | 0.06 |
| 2 | −0.02 | −0.06 | 0.06 | 0.09 | 0.05 | 0.05 |
| 3 | −0.03 | −0.07 | 0.04 | 0.11 | 0.04 | 0.04 |
| 4 | −0.03 | −0.06 | 0.07 | 0.07 | 0.03 | 0.06 |
| 5 | −0.06 | −0.06 | 0.08 | 0.09 | 0.04 | 0.05 |
| 6 | −0.02 | −0.05 | 0.05 | 0.08 | 0.05 | 0.05 |
| 7 | −0.03 | −0.06 | 0.06 | 0.1 | 0.04 | 0.05 |
| 8 | −0.05 | −0.04 | 0.04 | 0.07 | 0.03 | 0.04 |
| 9 | −0.07 | −0.05 | 0.05 | 0.08 | 0.05 | 0.05 |
| 10 | −0.06 | −0.09 | 0.09 | 0.09 | 0.04 | 0.06 |

### <Experimental Example 2> Evaluation of Alcohol Hangover Severity Scale. AHSS

Ten male and female subjects were 30 to 50 years old and weighed 65 to 75 kg, and alcohol intake was 400 ml (alcohol content 19.5%). An experiment was conducted to evaluate the symptoms felt before drinking and on the day after drinking (0=none, 10-extreme). The results are shown in Table 6 (after taking:before taking).

TABLE 6

| Classification | Subject 1 | Subject 2 | Subject 3 | Subject 4 | Subject 5 | Subject 6 | Subject 7 |
|---|---|---|---|---|---|---|---|
| Fatigue | 1:8 | 3:10 | 2:8 | 2:6 | 3:5 | 5:10 | 3:7 |
| Indifference | 0:10 | 0:9 | 0:5 | 0:0 | 1:5 | 3:10 | 1:3 |
| (less interest) | | | | | | | |
| Concentration | 0:9 | 2:10 | 1:3 | 1:4 | 1:5 | 0:5 | 0:5 |
| Issue | | | | | | | |
| Clumsiness | 0:8 | 2:9 | 0:3 | 2:3 | 1:4 | 0:0 | 0:3 |
| Confused | 0:10 | 2:9 | 0:5 | 4:6 | 1:2 | 3:3 | 0:1 |
| (distracted) | | | | | | | |
| Thirst | 3:10 | 3:10 | 3:5 | 3:7 | 4:6 | 0:7 | 2:8 |
| Sweating | 0:8 | 1:8 | 0:0 | 0:0 | 1:5 | 0:0 | 0:0 |
| (perspiration) | | | | | | | |
| Tremor (chill) | 0:5 | 1:9 | 0:0 | 2:4 | 1:4 | 0:2 | 0:0 |
| Gastrointestinal | 2:9 | 3:9 | 1:5 | 1:6 | 2:4 | 0:0 | 0:2 |
| disorder | | | | | | | |
| Nausea | 0:9 | 3:9 | 0:3 | 1:7 | 2:5 | 0:3 | 0:9 |
| (sickness) | | | | | | | |
| Vertigo | 1:5 | 3:10 | 4:10 | 3:8 | 1:2 | 0:0 | 0:1 |
| Heart tremor | 2:8 | 1:8 | 0:5 | 3:6 | 0:0 | 0:0 | 0:8 |
| (palpitation) | | | | | | | |

From above Table 6, it can be seen that the degree of alcohol hangover severity after taking the health functional food according to the present invention is ameliorated.

### <Formulation Example 1> Preparation of Pill

Pill was prepared by a conventional method according to the composition shown in Table 7 below.

TABLE 7

| | Formulation | Formulation |
|---|---|---|
| Raw material | Example 1 (unit: wt %) | Example 1 (unit: g) |
| L-ornithine | 23 | 1.035 |
| Potassium citrate | 6.7 | 0.3015 |
| Cabbage powder | 35 | 1.575 |
| L. casei | Total 6 | Total 0.27 |
| L. plantarum | | |
| B. breve | | |
| L. acidophilus | | |
| L. rhamnosus | | |
| B. animalis ssp. Lactis | | |
| L. lactis | | |
| Oenanthe javanica powder | 8.14 | 0.3663 |
| Curcuma longa L extract | 8 | 0.36 |
| L-asparagine | 5 | 0.225 |
| Oriental raisin tree fruit extract | 3 | 0.135 |
| Vitamin-mineral mix | 1 | 0.045 |
| Niacin | 0.56 | 0.0252 |
| Licorice (from Uzbekistan) | 0.5 | 0.0225 |
| Oriental raisin tree fruit (domestic) | 0.5 | 0.0225 |
| Pueraria (domestic) | 0.5 | 0.0225 |
| Jujube (domestic) | 0.5 | 0.0225 |
| Xylooligosaccharide | 0.1 | 0.0045 |
| Fructooligosaccharide | 0.1 | 0.0045 |
| Milk thistle | 0.1 | 0.0045 |
| Vitamin B1 | 0.1 | 0.0045 |
| Taurine | 0.1 | 0.0045 |
| Aminomix-9 | Total 0.1 | Total 0.0045 |
| (L-glutamine, L-leucine, L- | | |
| phenylalanine, | | |
| L-lysine hydrochloride, L-valine, | | |
| L-threonine, L-isoleucine, L- | | |
| methionine, | | |
| L-tryptophan) | | |

### <Formulation Example 2> Preparation of Pill

Pill was prepared by a conventional method according to the composition shown in Table 8 below.

TABLE 8

| Raw material (unit: g) | Formulation example 2 | Formulation example 3 |
|---|---|---|
| L-ornithine | 1.035 | 1.349 |
| Potassium citrate | 0.3015 | 0.392 |
| Cabbage powder | 1.8 | 2.34 |
| B. breve | 0.045 | Total 0.41 |
| L. casei | Total 0.27 | |
| L. plantarum | | |
| L. acidophilus | | |
| L. rhamnosus | | |
| B. animalis ssp. | | |
| Lactis | | |
| L. lactis | | |

### <Formulation Example 9> Preparation of Pill

A health functional beverage was prepared by a conventional method according to the composition shown in Table 9 below.

TABLE 9

| Raw material | Formulation | Formulation | Formulation | Formulation |
|---|---|---|---|---|
| (unit: g) | example 4 | example 5 | example 6 | example 7 |
| L-ornithine | 3 | 3 | 3 | 3 |
| Potassium | 1 | 1 | 1 | 1 |
| citrate | | | | |
| Cabbage | 1.1 | 16 | 2.6 | 4.1 |
| powder | | | | |
| B. breve | 0.05~0.5 | 0.05~0.5 | 0.05~0.5 | 0.05~0.5 |
| L. casei | | | | |
| L. plantarum | | | | |
| L. acidophilus | | | | |
| L. rhamnosus | | | | |
| B. animalis | | | | |
| ssp. | | | | |
| Lactis | | | | |
| L. lactis | | | | |

While the invention has been described in connection with the preferred exemplary embodiments mentioned above, it is possible to make various modifications or variations without departing from the spirit and scope of the invention. Accordingly, the appended claims will include such modifications or variations as long as they fall within the subject matter of the present invention.

What is claimed is:

1. A food composition for hangover relief and liver function improvement, comprising:
L-ornithine, potassium citrate, cabbage powder, and one or more lactic acid bacteria selected from the group consisting of bifidobacteria and lactobacillus.

2. The food composition of claim 1, wherein the L-ornithine is contained in an amount of 15 to 30 wt % based on the total weight of the composition,
the potassium citrate is contained in an amount of 2 to 12 wt % based on the total weight of the composition,
the cabbage powder is contained in an amount of 30 to 50 wt % based on the total weight of the composition, and
the lactic acid bacteria are contained in an amount of 2 to 12 wt % based on the total weight of the composition.

3. A method for preparing a food composition for hangover relief and liver function improvement, the method comprising:
preparing a mixture by mixing a raw powder material including L-ornithine, potassium citrate, cabbage powder, and one or more lactic acid bacteria selected from the group consisting of bifidobacteria and lactobacillus with water;
making the mixture into pill to prepare a pill precursor;
molding the pill precursor to smooth a surface thereof; and
drying the pill precursor having the smoothly molded surface to prepare pill.
